(19) United States
(12) Patent Application Publication
(10) Pub. No.: US 2005/0026927 A1
(43) Pub. Date: Feb. 3, 2005

(54) Pyrazole derivatives as psychopharmaceuticals

(76) Inventors: Henning Boettcher, Darmstadt (DE); Christoph Seyfried, Seeheim-Jugenheim (DE); Christoph Van Amsterdam, Darmstadt (DE); Gerd Bartoszyk, Weiterstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/495,456

(22) PCT Filed: Oct. 14, 2002
(86) PCT No.: PCT/EP02/11464

(30) Foreign Application Priority Data
Nov. 14, 2001 (EP) 01126531.1

(51) Int. Cl. C 07D 4 3/14; A61K 31/496

(52) U.S. Cl. 514/253.06; 514/254.05; 544/363; 544/371

(57) ABSTRACT

The invention relates to pyrazole derivatives of Formula (I), in which R?1 [symbol] , X, Z and n have the meanings indicated above, their preparation and their use as psychopharmaceuticals and/or as active compounds of medicaments for the treatment and prophylaxis of movement disorders and/or for the manufacture of a medicament for the treatment of adverse effects of anti-Parkinsonian drugs in extrapyramidal movement disorders and/or for the manufacture of a medicament for the treatment of extrapyramidal symptoms (EPS) induced by neuroleptics.

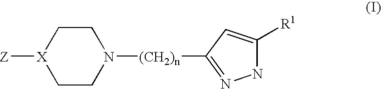

The invention relates to pyrazole derivatives, their preparation and their use as psychopharmaceuticals and/or as active compounds of medicaments for the treatment and prophylaxis of movement disorders and/or for the manufacture of a medicament for the treatment of adverse effects of anti-Parkinsonian drugs in extrapyramidal movement disorders and/or for the manufacture of a medicament for the treatment of extrapyramidal symptoms (EPS) induced by neuroleptics.

The pyrazole derivatives according to the invention can be represented by the general formula I 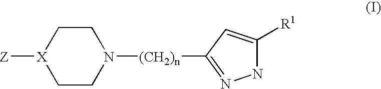
where
X is N or CH,
Z is 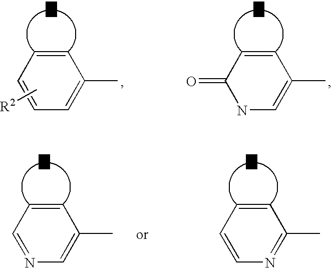
A is an aromatic or aliphatic ring wherein one or more CH-groups may be replaced by N or CR², or wherein one or more CH₂-groups may be replaced by NH, CO, SO, SO₂, S or O,
R¹ is H, or alkyl having 1 to 10 C-atoms,
R² is H, Halogen or alkyl or alkoxy having 1 to 10 C-Atoms, wherein one or more H-atoms may be replaced by F,
   and
n is 1, 2, 3 or4
and their salts and solvates, preferably their physiologically acceptable salts and solvates.

Psychoses, which also include diseases of the schizophrenia type, have been attributed to a hyperactivity of the limbic dopamine system (Snyder et al., Science 184: 1243-1253, 1974). The antipsychotic effect of neuroleptics has been attributed to their D2-antagonistic properties (with regard to the nomenclature of the receptors: Basic Neurochemistry, Editors: G. J. Siegel, B. W. Agranoff, R. W. Albers, P. B. Molinoff, 5th edition, Raven Press, Ltd, New York USA, Chapters 12 and 13; otherwise the following technical publications: Creese et al., Science 192: 481-483, 1976; Farde et al., Psychopharmacology 99: 28-31, 1989; Feeman et al., Nature 261: 717-719, 1976; Wiesel et al., Prog. Neuro-Psychopharmacol. & Biol. Psychiat. 14: 759-767, 1990). Consequently, the classical dopamine hypothesis of schizophrenia was formulated, according to which neuroleptics have to bind to the D₂ receptor. On account of their extrapyramidal side effects, the employment of classical D₂ antagonists is severely restricted, especially in the case of chronic administration. The extrapyramidal side effects include, for example, tremor, akinesia, dystonia and akathisia (Cavallaro & Smeraldi, CNS Drugs 4: 278-293, 1995). There are only a few antipsychotics which cause significantly fewer or no extrapyramidal side effects at all and which are described as “atypical neuroleptics” (Kervin, Brit. J. Psychiatry 1964, 141-148, 1994). The prototype atypical neuroleptic clozapine has extremely low extrapyramidal side effects, but causes other serious complications such as agranulocytosis, which sometimes is fatal (Alvir et al., New Engl. J. Med. 329: 162-167, 1993).

Because 5-HT_{1A} agonists intensify antipsychotic properties of conventional dopamine D₂ antagonists in animals (Wadenberg & Ahlenios, J. Neural. Transm. 74: 195-198, 1988) and prevent the catalepsy induced by dopamine D₂ antagonists (Costall et al., Neuropharmacology 14: 859-868, 1975), 5-HT_{1A}-agonistic properties could be advantageous. The efficacy of buspirone, a pharmacon having 5-HT_{1A}-agonistic and dopamine D₂-antagonistic properties, has been demonstrated in schizophrenia patients (Goff et al., J. Clin, Psychopharmacol. 11: 193-197, 1991). Apart from various dopamine autoreceptor agonists which also have a significant affinity for the 5-HT_{1A} receptor (e.g. U-86170F, Lahti et al., Naunyn-Schmiedeberg's Arch. Pharmacol. 344: 509-513, 1991), PD1431188 (Meizer et al., J. Pharmacol. Exp. Ther. 274: 912-920, 1995) and roxindole (Bartoszyk et al., J. Pharmacol., Exp. Ther. 276: 41-48, 1996), only a few dopamine D₂ antagonists have been developed which also have an affinity for the 5-HT_{1A} receptor, such as mazapertine (Reiz et al., J. Med. Chem. 37: 1060-1062, 1994), S16924 (Millan et al., Br. J. Pharmacol. 114: 156 B, 1995) or ziprasidone (Seeger et al., J. Pharmacol. Exp. Ther. 275: 101-113, 1995). These already known compounds have disadvantages with respect to affinity or specificity. Thus mazapertine also shows an affinity for the α₁ receptor. S16924 additionally has 5-HT_{2A/C}-antagonistic properties and ziprasidone moreover binds to the 5-HT_{1D/2A/2C} receptors.

It is the object of the invention to make available medicaments, in particular psychopharmaceuticals. It is a further object of the invention to make available compounds which bind both to the dopamine D₂ receptor and to the 5-HT_{1A} receptor.

This object is achieved by the compounds of the general formula I and by their tolerable salts and solvates

It has been found that the compounds of the formula I and their salts and solvates have very valuable pharmacological properties together with good tolerability. They especially act on the central nervous system. They have, in particular, a high affinity for receptors of the 5-HT_{1A} type and/or of the dopamine D2 type.

Compounds of the formula I are particularly preferably simultaneously agonists of the 5-HT_{1A} receptor and antagonists of the D₂ receptor. Binding to additional 5-HT_{1D/2A/2C} receptors is not observed.

Binding properties of the compounds of the formula I can be determined by known 5-HT_{1A} (serotonin) binding test and dopamine binding tests; (5-HT_{1A} (serotonin) binding test: Matzen et al., J. Med. Chem., 43, 1149-1157, (2000) in particular page 1156 with reference to Eur. J. Pharmacol.: 140, 143-155 (1987); dopamine binding tests: Böttcher et al., J. Med. Chem.: 35, 4020-4026, (1992) with reference to J. Neurochem.: 46, 1058-1067 (1986).

The compounds of the formula I differ from the abovementioned atypical neuroleptics.

The compounds according to the invention can be employed for the treatment and prophylaxis of diseases which are associated with the serotinin and dopamine neurotransmitter system and in which high-affinity serotinin receptors (5-HT_{1A} receptors) and/or dopamine D₂ receptors are involved. The most important indication for the administration of the compound of the general formula I are psychoses of any type, in particular also mental disorders of the schizophrenia type. Moreover, the compounds can also be employed for the reduction of cognitive functional disorders, i.e. for improvement of the learning ability and of the memory. The compounds of the general formula I are also suitable for the control of the symptoms of Alzheimer's disease. The substances of the general formula I according to the invention are moreover suitable for the prophylaxis and control of cerebral infarcts (cerebral apoplexy), such as cerebral stroke and cerebral ischaemia. The substances are also suitable for the treatment and prohylaxis of disorders such as pathological anxiety states, overexcitation, hyperactivity and attention disorders in children and adolescents, developmental disorders and disorders of social behaviour with mental retardation, depression, compulsive disorders in the narrower (OCD) and wider sense (OCSD), certain sexual function disorders, sleep disorders and eating disorders, and also such psychiatric symptoms in the context of senile dementia and dementia of the Alzheimer type, i.e. diseases of the central nervous system in the widest sense.

They can be furthermore used for treating side-effects in the treatment of hypertension, in endocrinology and gynecology, e.g. for the treatment of acromegaly, hypogonadism, secondary amenorrhea, premenstrual syndrome or undesired puerperal lactation.

Furthermore, it has been found that the compounds of the present invention or physiologically acceptable salts and solvates thereof also have therapeutic activity against extrapyramidal movement disorders such as idiopathic Parkinsons's disease, Parkinson syndromes, dyskinetic, choreatic, or dystonic syndromes, tremor, Gilles de la Tourette syndrome, ballism, myoclonus, restless legs syndrome or Wilsons's disease, as well as extrapyramidal motoric disturbances [synonymous extrapyramidal symptoms (EPS)] induced by neuroleptics.

Additionally, it has been found that the inventive compounds and their physiologically acceptable salts and solvates have therapeutic activity against adverse effects of anti-Parkinsonian drugs in extrapyramidal movement disorders, in particular against dopaminomimetic adverse effects of anti-Parkinsonian drugs in idiopathic Parkinson's disease or Parkinson syndromes.

Furthermore, it has been found that the compounds of the present invention and their physiologically acceptable salts and solvates show an extremely low liability to induce extrapyramidal side effects. Extrapyramidal motor side effects in e.g. rodents are measured by the ability of a drug to induce catalepsy. Catalepsy is defined as a state where an animal continues to remain in an unnormal (nonphysiological ‘uncomfortable’) posture for a long time (e.g.: M. E. Stanley and S. D. Glick, Neuropharmacology, 1996; 15: 393-394; C. J. E. Niemegeers and P. Janssen, Life Sci., 1979, 201-2216). For example, if a hindpaw of a rat is placed on an elevated level, e.g. a platform elevated 3 cm above ground level, a normal rat immediately withdraws the hindpaw from the platform to the ground level. A cataleptic rat remains in this unnatural posture even for minutes.

Although the compounds of formula I and their physiologically acceptable salts and solvates have a dopamine antagonistic mechanism of action which is known to induce extrapyramidal motor side effects (C. J. E. Niemegeers and P. Janssen, Life Sci., 1979, 201-2216), unexpectely the compounds of formula I do not induce any catalepsy in rats in even higher doses than those effective in the animal models indicative for the before-mentioned therapeutic indications.

Even more unexpectedly, the compounds of formula I and their physiologically acceptable salts and solvates moreover are able to prevent catalepsy induced by conventional antidopaminergic drugs and even reverses already existing catalepsy induced by conventional antidopaminergic drugs such as haloperidol; the doses for this anticataleptic effect are in the same dose-range shown to be effective in the animal models indicative for the before-mentioned therapeutic indications.

Beneficial effects on the extrapyramidal motoric system have previously been described for other drugs with 5-HT_{1A} agonistic action. Buspirone for example, which is an anxiolytic drug by nature, exhibits moderate anti-dyskinetic properties in advanced Parkinson patients (B. Kleedorfer et al., J Neurol Neurosurg Psychiatry, 1991, 54: 376-377; V. Bonifati et al., Clin Neuropharmacol, 1994, 17: 73-82). The main mechanism of action is obviously via stimulation of 5-HT_{1A} receptors of the raphe nigral and raphe striatal pathways. In contrast to buspirone, the compounds of formula I and their physiologically acceptable salts and solvates are by far more potent agonists at the 5-HT_{1A} receptor.

Furthermore, the compounds of formula I and their physiologically acceptable salts and solvates exhibit a D₂ antagonism under increased doses which represents an additional advantage in comparison to conventional 5-HT_{1A} agonists like buspirone. On one hand, the D₂ antagonism lowers the risk of psychotic reactions caused by the stimulation of serotonin receptors and, on the other hand, emphasises indirectly the D₁ properties of the co-administered non-selective D₁/D₂ agonist l-dopa. A more selective stimulation of D₁ receptors is known to be beneficial for the treatment of dyskinesias in Parkinson's disease (P. J. Blanchet et al., J Neural Transm, 1995, 45 (Suppl.): 103-112). Therefore both, the 5-HT_{1A} agonistic and the D₂ antagonistic properties of the compounds of formula I or a physiologically acceptable salt or solvate thereof, contribute to the advantageous effects on the extrapyramidal motoric system.

The pharmacological profile of the compounds of formula I and their physiologically acceptable salts and solvates are furthermore characterized by a high affinity to the dopamine D₃ receptor. The D₃ receptor is obviously involved in the pathogenesis of dyskinesia. An association between a genetic polymorphism of the dopamine D₃ receptor and the disposition to develop tardive dyskinesia has recently been reported (Segmann et al. 1999, Mol-Psychiatry 4: 247). Additionally, there is obviously an increased density of dopamine D₃ receptors in Parkinson patients with l-dopa-induced dyskinesia. Therefore, the interaction of the compounds of formula I or one of their physiologically acceptable salts and solvates with the dopamine D₃ receptor is an additional important mechanism leading to beneficial effects on the extrapyramidal system, in particular in the treatment of dyskinesia.

The atypical neuroleptic clozapine is regarding the extrapyramidal effects—but not regarding structure or side effects—congruent with the compounds of formula I and their physiologically acceptable salts and solvates particularly in scope of the anticataleptic properties. Recent studies provide evidence that clozapine ameliorates dyskinesias in Parkinson's disease (F. Perelli et al., Acta Neurol Scan, 1998, 97: 295-299; P. Pollak et al., Lancet, 1999, 353: 2041-2041). Besides that, clozapine is known to have a variety of other beneficial effects on extrapyramidal movement disorders, like in tardive dyskinesia, tremor, Huntington's disease, Tourette's syndrome, akathisia and dopaminomimetic psychosis (C. Pfeiffer and M. L. Wagner, Am J Hosp Pharm, 1994, 51: 3047-3053). The compounds of formula I and their physiologically acceptable salts and solvates improve these kinds of movement disorders even without bearing the risk of the fatal side effects of clozapine like agranulocytosis and acute nephritis (J. Alvir et al., N Engl J Med, 1993, 329: 162-167; T. J. Elias et al., Lancet, 1999, 354: 1180-1181).

Therefore, the present invention relates to the use of the compounds of formula I or a physiologically acceptable salt or solvate thereof, for the manufacture of a medicament for the treatment of extrapyramidal movement disorders.

Especially preferred salts of the compounds of the formula I are the hydrochlorides.

Additionally, the invention relates to the use of a pharmaceutical composition containing at least one of the compounds of the present invention or one of its biocompatible salts and solvates together with at least one solid, liquid or semiliquid excipient or adjunct for the treatment of extrapyramidal movement disorders.

The inventive compounds of formula I and their physiologically acceptable salts and solvates are especially useful for the treatment of extrapyramidal movement disorders, in particular for the treatment of idiopathic Parkinson's disease, Parkinson syndromes, dyskinetic, choreatic or dystonic syndromes, extrapyramidal motoric adverse effects of neuroleptics, tremor, Gilles de la Tourette syndrome, ballism, myoclonus, restless legs syndrome or Wilson's disease and/or useful for the treatment of adverse effects in idiopathic Parkinson's disease or Parkinson syndromes including medicinal compositions as defined below, are preferably administered in doses from 0.1 to 100 mg, preferentially between approximately 1 and 20 mg. The composition may be administered once or more times a day, e.g. 2, 3, or 4 times daily. The specific dose for each patient depends on all sorts of factors, e.g. on the activity of the specific compound employed, on the age, body weight, general state of health, on sex, diet, time and route of administration, on the excretion rate, pharmaceutical substance combination and on the severity of the particular disorder to which the therapy relates. Oral administration is preferred, but also parenteral routes of administration (e.g. intravenous or transdermal) can be utilized.

Anti-Parkinsonian drugs are conventional drugs such as l-dopa (levodopa) and l-dopa combined with benserazide or carbidopa, dopamine agonists such as bromocriptine, apomorphine, cabergoline, pramipexol, ropinirol, pergolide, dihydro-α-ergocriptine or lisuride plus all drugs acting via stimulation of dopamine receptors, inhibitors of catechol-O-methyl transferase (COMT) such as entacapone or tolcapone, inhibitors of monoamine oxidase (MAO) such as selegiline and antagonists of N-methyl-D-aspartate (NMDA) receptors such as amantadine or budipine.

Adverse effects of said anti-Parkinsonian drugs are all types of dyskinesias, such as choreic, dystonic, ballistic and myoclonic dyskinesia, as well as motor (response) fluctuations or psychotic states.

Therefore, the present invention relates to the use of the compounds of formula I and their physiologically acceptable salts and solvates, for the manufacture of a medicament for the treatment of adverse effects of anti-Parkinsonian drugs in idiopathic Parkinson's disease.

Treatment of adverse effects of conventional anti-Parkinsonian drugs as defined above are determined in a modification of the animal model of the Parkinsonian cynomolgus monkey according to P. J. Blanchet et al., Exp. Neurology 1998; 153: 214-222. Monkeys render parkinsonian by repeated injections of 1-methyl-4-phenyl-1,2,3,6-tetrahydropyridine (MPTP). The Parkinsonian monkeys are chronically treated with the standard l-dopa therapy according to P. J. Blanchet et al., Mov. Disord., 1998; 13: 798-802. Longterm treatment with l-dopa induces extrapyramidal motor side effects and psychotic states which are both qualitatively and quantitatively, assessed by the Abnormal Involuntary Movement Scale (P. J. Blanchet et al., Mov. Disord. 1998; 13: 798-802) for different body parts (face, neck, trunk, each limb) and by rating for psychotic states by observing the monkey's attention, reactivity and mobility. The compounds of formula I reduced overall choreiform dyskinesias and dystonic dyskinesias as well as psychotic states.

Additionally, the invention relates to the use of a pharmaceutical composition containing at least one compound of formula I and/or one of their biocompatible salts and/or solvates together with at least one solid, liquid or semiliquid excipient or adjunct for the treatment of adverse effects of anti-Parkinsonian drugs in idiopathic Parkinson's disease.

Furthermore, the present invention relates to the use of the compounds of formula I and their physiologically acceptable salts and solvates, for the manufacture of a medicament for the treatment of idiopathic Parkinson's disease.

A typical animal model for idiopathic Parkinson's disease is the Parkinsonian cynomolgus monkey according to P. J. Blanchet et al., Exp. Neurology 1998; 153: 214-222.

Parkinsonian symptoms are qualitatively assessed by the use of the Laval University Disability Scale (B. Gomez-Mancilla et al., 1993; Mov. Disord. 8: 144-150) measuring the following symptoms: posture, mobility, climbing, gait, holding food, vocalizing, grooming, social interaction. The compounds of formula I and their physiologically acceptable salts and solvates reduced all the parkinsonian symptoms and increased total activity.

Additionally, the invention relates to the use of a pharmaceutical composition containing at least one compound of formula I and/or one of their biocompatible salts and/or solvates together with at least one solid, liquid or semiliquid excipient or adjunct for the treatment of idiopathic Parkinson's disease.

The limiting factor of Parkinson treatment with l-dopa and/or dopamine agonists is often the occurence of psychosis or dyskinesia and other motor fluctuations.

It has been found that the compounds of formula I and their physiologically acceptable salts and solvates enhance the anti-Parkinsonian effect of anti-Parkinsonian drugs as defined above without inducing extrapyramidal side effects.

Therefore, the add-on therapy with the inventive compounds or a physiologically acceptable salt or solvate thereof, now opens the possibility to increase the doses of l-dopa and/or dopamine agonists and/or all other anti-Parkinsonian drugs as defined above in order to counteract periods of insufficient motility (“off” phases) without provoking the above mentioned side effects. That represents an entirely novel approach in the treatment of Parkinson's disease leading to a significant benefit for the patients.

Thus, the invention relates to a pharmaceutical composition comprising, as active principles, (i) at least one compound of formula I or a physiologically acceptable salt or solvate thereof, and (ii) at least one anti-Parkinsonian drug, in combination with one or more pharmaceutically acceptable excipients.

Particularly, the invention relates to a pharmaceutical composition comprising, as active principles, (i) at least one compound of formula I or a physiologically acceptable salt or solvate thereof, and (ii) l-dopa or l-dopa combined with benserazide or carbidopa, in combination with one or more pharmaceutically acceptable excipients.

The ratios of the respective amounts of the compounds of formula I and/or their physiologically acceptable salts and/or solvates and of the conventional anti-Parkinsonian drug thus vary in consequences. Preferably, the weight ratio of the compounds of formula I and/or their physiologically acceptable salts and/or solvates to the conventional anti-Parkinsonian drug ranges from 1:1 to 1:100, preferably from 1:10 to 1:90 and better still from 1:40 to 1:60.

Another subject of the present invention is the use of the compounds of formula I and/or their physiologically acceptable salts and/or solvates in combination with at least one anti-Parkinsonian drug, for the preparation of a medicinal combination intended to enhance the anti-Parkinsonian effect of said anti-Parkinsonian drugs.

According to the invention, the term “medicinal combination” is intended to refer either to a pharmaceutical composition as defined above, in which the two active principles or compounds are the essential constituents of the same composition, or to a kit comprising two separate compositions, the first comprising at least one of the compounds of formula I and/or their physiologically acceptable salts and/or solvates as sole active principle, and the second comprising at least one anti-Parkinsonian drug as active compound.

When the medicinal combination is in the form of a kit, the administration of the two compositions constituting this kit, although carried out separately, is simultaneous for a combined therapy.

Adverse effects of anti-Parkinsonian drugs as defined above are additionally known in particular in Parkinson syndromes.

Parkinson syndromes are e.g. multiple system atrophies (MSA), Steele-Richardson-Olszewski syndrome (=progressive supranuclear palsy), cortico-basal degeneration, olivo-ponto cerebellar atrophy or Shy Drager syndrome.

The compounds of formula I and their physiologically acceptable salts and solvates are useful for the treatment of Parkinson syndromes in particular of multiple system atrophies.

Therefore the present invention relates to the use of the compounds of formula I and their physiologically acceptable salts and solvates, for the manufacture of a medicament for the treatment of adverse effects in Parkinson syndromes.

The present invention relates additionally to the use of the compounds of formula I and their physiologically acceptable salts and solvates, for the manufacture of a medicament for the treatment of Parkinson syndromes.

A typical animal model is the reserpinized rat or mouse (e.g. M. S. Starr and B. S. Starr, J. Neural Transm.—Park. Dis. Dement. Sect., 1994; 7: 133-142; M. Gossel et al., J. Neural Transm.—Park. Dis. Dement. Sect., 1995; 10: 27-39; N. R. Hughes et al., Mov. Disord., 1998; 13: 228-233). Reserpine is a potent depleter of monoamines and produces nearly complete akinesia in both species. Prominent 24 h after application, the distance travelled and the time active is nearly zero as measured in conventional activity meters. The compounds of formula I and their physiologically acceptable salts and solvates dose-dependently reduced akinesia, i.e. restored distance travelled and time active to about the level of normal animals.

Another more recent animal model is the striatonigral degeneration approach in the rat according to G. K. Wenning et al., J. Neural Transm. Suppl., 1999; 55: 103-113. Rats receive an unilateral injection of 6-hydroxydopamine into the left medial forebrain bundle followed by an injection of quinolinic acid into the ipsilateral striatum inducing nigrostriatal degeneration. The degeneration results in turning behavior to a challenge with dopaminomimetics such as apomorphine or amphetamine. Turning behavior is measured by an automated recorder. Turning behavior induced by apomorphine or amphetamine was dose-dependently antagonized by the compounds of formula I and their physiologically acceptable salts or solvates.

Multiple system atrophy (MSA) is due to an expansive neurodegeneration in the extrapyramidal and autonomic nervous system which leads to an akinetic Parkinsonian syndrome with vegetative disturbances. In contrast to idiopathic Parkinson's disease the density of central dopamine receptors is markedly decreased and therefore, MSA patients poorly respond to dopaminergic drugs. Since the compounds of formula I and their physiologically acceptable salts or solvates act predominantly via serotonin receptors on the extrapyramidal system, they are able to improve the motor performance in these otherwise mostly untreatable patients.

Therefore, the invention relates to the use of the compounds of formula I and their physiologically acceptable salts or solvates for the manufacture of a medicament for the treatment of adverse effects of anti-Parkinsonian drugs in Parkinson syndromes.

Additionally, the invention relates to the use of a pharmaceutical composition containing at least one compound of formula I or one of its biocompatible salts or solvates together with at least one solid, liquid or semiliquid excipient or adjunct for the treatment of adverse effects of anti-Parkinsonian drugs in Parkinson syndromes.

Additionally, the invention relates to the use of a pharmaceutical composition containing at least one compound of of formula I or one of its biocompatible salts or solvates together with at least one solid, liquid or semiliquid excipient or adjunct for the treatment of Parkinson syndromes.

The present invention relates to the use of the compounds of formula I and their physiologically acceptable salts or solvates, for the manufacture of a medicament for the treatment of dyskinetic and/or choreatic syndromes.

Dyskinetic and/or choreatic syndromes are e.g. Huntington's disease, minor chorea or chorea of pregnancy.

The compounds of formula I and their physiologically acceptable salts or solvates are in particular useful for the treatment of Huntington's disease.

A typical animal model is the systemic 3-nitropropionic acid (3-NP) model in rats according to C. V. Borlongan et al, Brain Res., 1995; 697: 254-257. Rats are treated with injections of the selective striatal neurotoxin 3-NP i.p. every fourth day (C. V. Borlongan et al., Brain Res. Protocols, 1997; 1: 253-257). After two injections of 3-NP, rats display nocturnal hyperactivity reflecting symptoms of early Huntington's disease, whereas rats treated with four injections of 3-NP display nocturnal akinesia (hypoactivity) reflecting symptoms of late Huntington's disease. Nocturnal activity is automatically measured in conventional activity cages by infrared beams. The compounds of formula I and their physiologically acceptable salts or solvates reduced both the nocturnal hyperactivity and akinesia.

Therefore, the invention relates to the use of the compounds of formula I and their physiologically acceptable salts or solvates for the manufacture of a medicament for the treatment of dyskinetic and/or choreatic syndromes, in particular for the treatment of Huntington's disease.

Additionally, the invention relates to the use of a pharmaceutical composition containing at least one compound of formula I or one of its biocompatible salts or solvates together with at least one solid, liquid or semiliquid excipient or adjunct for the treatment of dyskinetic and/or choreatic syndromes.

Dystonic syndromes are e.g. spasmalic torticollis, writer's cramp, blepharospasm, Meige syndrome or dopasensitive dystonia.

The compounds of formula I and their physiologically acceptable salts or solvates are in particular useful for the treatment of spasmalic torticollis and/or blepharospasm.

A typical animal model is the mutant dystonic hamster according to A. Richter and W. Löscher, Prog. Neurobiol. 1998; 54: 633-677. In this genetically dystonic hamsters, dystonic attacks are provoked by taking the animal from the home cage and placing it on a balance. The dystonic syndrome consists of a sequence of abnormal movements, and the severity of the single symptoms is rated by a scoring system. The compounds of formula I and their physiologically acceptable salts or solvates dose-dependently reduce the severity of dystonic symptoms.

Therefore the invention relates to the use of the compounds of formula I and their physiologically acceptable salts or solvates for the manufacture of a medicament for the treatment of dystonic syndromes, in particular of spasmalic torticollis and/or blepharospasm.

Additionally, the invention relates to the use of a pharmaceutical composition containing at least one compound of formula I or one of its biocompatible salts or solvates together with at least one solid, liquid or semiliquid excipient or adjunct for the treatment of dystonic syndromes.

The present invention relates to the use of compounds of formula I and their physiologically acceptable salts or solvates, for the manufacture of a medicament for the treatment of extrapyramidal symptoms induced by neuroleptics.

Extrapyramidal motoric disturbances induced by neuroleptics are e.g. early dyskinesia, dystonia, akathisia, parkinsonoid, in particular bradykinesia, or tardive dyskinesia.

The compounds of formula I and their physiologically acceptable salts or solvates are useful particularly for the treatment of akathisia and/or tardive dyskinesia and/or parkinsonoid.

A typical animal model is neuroleptics-induced muscle rigidity in rats according to S. Wolfarth et al., Arch. Pharmacol. 1992; 345: 209-212. Rats are challenged with the conventional neuroleptic drug haloperidol which enhances muscle tone. Muscle tone is electromechanically measured as the resistence to passive flexion and extension of the hind limb. The compounds of formula I and their physiologically acceptable salts or solvates decreased the muscle tone enhanced by haloperidol.

Another typical animal model is the neuroleptics sensitized monkey according to D. E. Casey, Psychopharmacology, 1996; 124: 134-140. Monkeys treated repeatedly with conventional neuroleptics are highly sensitive to a subsequent challenge dose of neuroleptic drugs. When challenged, the monkeys immediately show extrapyramidal motor side effects such as dystonia, dyskinesias, akathisia, and bradykinesia which are rated by a scoring system. The conventional neuroleptic drug haloperidol is given as a challenge. When the before-mentioned extrapyramidal motor side effects occur, a compound of formula I or its physiologically acceptable salts or solvates is administered; The inventive compounds dose-dependently reduce the extrapyramidal motor side effects.

Tardive dyskinesia is a common adverse effect of long-term treatment with neuroleptics.

Therefore, the invention relates to the use of the compounds of formula I and their physiologically acceptable salts or solvates for the manufacture of a medicament for the treatment of extrapyramidal symptoms induced by neuroleptics, in particular of akathisia and/or tardive dyskinesia.

Additionally, the invention relates to the use of a pharmaceutical composition containing at least one compound of formula I or one of its biocompatible salts or solvates together with at least one solid, liquid or semiliquid excipient or adjunct for the treatment of extrapyramidal symptoms induced by neuroleptics.

The compounds of the present invention and their salts and solvates are also useful in the treatment of tremor.

Tremor includes all types of tremors such as essential tremor, activated physiological tremor, cerebellar tremor, orthostatic tremor or drug-induced tremor.

The compounds of formula I and their physiologically acceptable salts or solvates are particularly useful for the treatment of essential tremor and/or drug-induced tremor.

Typical animal models utilize either genetic mutant animals or are models where tremor is induced by a pharmacological agent (for review: H. Wilms et al., Mov. Disord., 1999; 14: 557-571).

Typical genetic models in mutant animals are the Campus Syndrome in the Pietrain pig according to A. Richter et al. (Exp. Neurology, 1995; 134: 205-213) or the Weaver mutant mouse according to J. R. Simon and B. Ghetti (Mol. Neurobiol., 1994; 9: 183-189). In the Campus Syndrome model, these mutant pigs show a high-frequency tremor when standing and during locomotion, but not while lying at rest. Assessment of tremor is made by accelerometric recording. In the Weaver mutant mouse, degenerative cerebellar atrophy is found in association with tremor, gait instability, and toppling over the sides after a few steps. Gait disability and toppling result in dramatically reduced locomotor activity measured by the distance travelled and the time spent with ambulation in conventional activity cages.

The compounds of formula I or one of its pharmaceutically acceptable salts or solvates improved the Campus Syndrome in the Pietrain pig, i.e. reduced disabling tremor when standing and during locomotion, and enhanced locomotor activity in the Weaver mutant mouse.

A typical animal model for drug-induced tremors is the oxotremorine-induced tremor (e.g. H. Hallberg and O. Almgren, Acta Physiol. Scand., 1987; 129: 407-13; J. G. Clement and W. R. Dyck, J. Pharmacol. Meth., 1989; 22: 25-36). Oxotremorine induces tremor which is measured by a rating scale. The compounds of formula I and their physiologically acceptable salts or solvates inhibit oxotremorine-induced tremors.

Therefore the invention relates to the use of the compounds of formula I and their physiologically acceptable salts or solvates for the manufacture of a medicament for the treatment of tremors, in particular of essential tremors and/or drug-induced tremors.

Additionally, the invention relates to the use of a pharmaceutical composition containing at least one compound of the formula I or one of its biocompatible salts or solvates together with at least one solid, liquid or semiliquid excipient or adjunct for the treatment of tremor.

The present invention relates to the use of the compounds of formula I or a physiologically acceptable salt or solvate thereof, for the manufacture of a medicament for the treatment of extrapyramidal movement disorders chosen from the group consisting of Gilles de la Tourette syndrome, ballism, myoclonus, restless legs syndrome and Wilson's disease.

A typical animal model for myoclonus is myoclonus induced by an acute hypoxic episode according to D. D. Truong et al., Mov. Dsiord., 1994; 9: 201-206). In this model of posthypoxic myoclonus, rats undergo a cardiac arrest for 8 minutes and are resuscitated thereafter. Myoclonic jerks occur spontaneously but can be provoked by auditory stimulation, too, worsening over the days following cardiac arrest. The compounds of formula I or one of its pharmacologically acceptable salts or solvates dose-dependently reduced the number of spontaneous and autitory-evoked myoclonic jerks.

Therefore the invention relates to the use of the compounds of formula I and their physiologically acceptable salts or solvates for the manufacture of a medicament for the treatment of extrapyramidal movement disorders chosen from the group consisting of Gilles de la Tourette syndrome, ballism, myoclonus, restless legs syndrome and Wilson's disease.

Additionally, the invention relates to the use of a pharmaceutical composition containing at least one compound of formula I or one of its biocompatible salts or solvates together with at least one solid, liquid or semiliquid excipient or adjunct for the treatment of extrapyramidal movement disorders chosen from the group consisting of Gilles de la Tourette syndrome, ballism, myoclonus, restless legs syndrome and Wilson's disease.

The extrapyramidal movement disorders such as Steele-Richardson-Olszewski syndrome (=progressive supranuclear palsy), cortico-basal degeneration, olivo-ponto cerebellar atrophy, Shy Drager syndrome, minor chorea, chorea of pregnancy, writer's cramp, blepharospasm, Meige syndrome, dopa-sensitive dystonia, Gilles de la Tourette syndrome, ballism, myoclonus, restless legs syndrome, and Wilson's disease are not frequent enough to perform regular double-blind trials. However, the medical need in this field is pressing since no sufficient therapies are available so far.

All the pharmaceutical preparations used for the treatment of extrapyramidal movement disorders and/or for the treatment of adverse effects of anti-Parkinsonian drugs in extrapyramidal movement disorders including the medicinal combination can be used as pharmaceuticals in human or veterinary medicine.

The compositions of the invention are preferably administered parenterally, or better still orally, although the other routes of administration, for instance such as rectal administration, are not excluded.

Suitable excipients are organic or inorganic substances which are suitable for enteral (e.g. oral), parenteral or topical adminstration and which do not react with the compounds of formula I and/or one of its biocompatible salts or solvates, for example water, vegetable oils, benzyl alcohols, alkylene glycols, polyethylene glycols, glycerol triacetate, gelatine, carbohydrates such as lactose or starch, magnesium stearate, talc, petroleum jelly. Forms which are used for oral administration are, in particular, tablets, pills, sugar-coated tablets, capsules, powders, granules, syrups, liquids or drops, forms for rectal administration are, in particular suppositories, forms for parenteral administration are, in particular, solvents, preferably oily or aqueous solutions, furthermore suspensions, emulsions or implants, and forms for topical administration are transdermal plasters, ointments, creams or powders. 1-[4-(5-cyanoindol-3-yl)butyl]4-(2-carbamoyl-benzofuran-5-yl)-piperazine and/or one of its pharrnaceutically acceptable salts or solvates may also be lyophilized and the resulting lyophilisates used for example for the preparation of injectable products. The above mentioned preparations can be in sterilized form and/or comprise auxiliaries such as glidants, preservatives, stabilizers and/or wetting agents, emulsifiers, salts for modifying the osmotic pressure, buffer substances, colourings, flavourings and/or other active ingredients, e.g. one or more vitamins.

Preparations may, if desired, be designed to give slow release of the compounds of formula I or a biocompatible salt or solvate thereof.

The compounds of the formula I and their physiologically acceptable salts or solvates are especially useful for the prophylaxis and treatment of pathological anxiety states, depression, psychoses and movement disorders such as Morbus Parkinson, dyskinesia or akathisia which may have been induced by neuroeptics or medicaments having a direct or indirect effect on the dopaminergic system.

The compounds of the formula I and their physiologically acceptable salts or solvates and solvates may be used as active ingredients for medicaments such as anxiolytics, antidepressants, neuroleptics, antihypertensives, antipsychotics and/or for medicaments for the prophylaxis and treatment of compulsive disorders, sleep disorders, dyskinesia, learnig disability and age-dependend memory disorders, eating disorders such as bulimie and/or sexual disorders.

Furthermore, the compounds of the formula I are useful as intermediates for the manufacture of active ingredients of medicaments.

Therefore, the present invention relates to the compounds of formula I and their salts or solvates and solvates and especially their physiologically acceptable salts or solvates and solvates and their use in human or veterinary medicine.

The compounds of the general formula I and their tolerable salts or solvates can thus be employed as active ingredients of medicaments such as anxiolytics, antidepressants, neuroleptics and/or antihypertensives.

R¹ is preferably H or alkyl having 1 to 6 C-atoms, where 1 to 7 hydrogen atoms are optionally replaced by fluorine. R¹ can be branched or unbranched and is preferably methyl, ethyl, propyl, isopropyl, n-butyl, sec-butyl, tert-butyl furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl. Particulary preferably R¹ is methyl.

R² is preferably h or alkoxy mit 1 bis 6 C-atomen. R² can be branched or unbranched and is preferably methoxy, ethoxy, n-propoxy, isopropoxy, n-butoxy, sec-butoxy, tert-butoxy furthermore also pentoxy, 1-, 2- or 3-methylbutoxy, 1,1-, 1,2- or 2,2-dimethylpropoxy, 1-ethylpropoxy, hexyloxy, 1-, 2-, 3- or 4-methylpentoxy, 1,1-, 1,2-, 1,3-, 2,2-, 2,3- or 3,3-dimethylbutoxy, 1- or 2-ethylbutoxy, 1-ethyl-1-methylpropoxy, 1-ethyl-2-methylpropoxy, 1,1,2- or 1,2,2-trimethylpropoxyParticulary preferably R² is methoxy or ethoxy, especially ethoxy.

X is preferably N.

The group Z preferably consists of 9 or 10 ring members, especially preferably of 9 ring members. Z is preferably chosen from the following group: 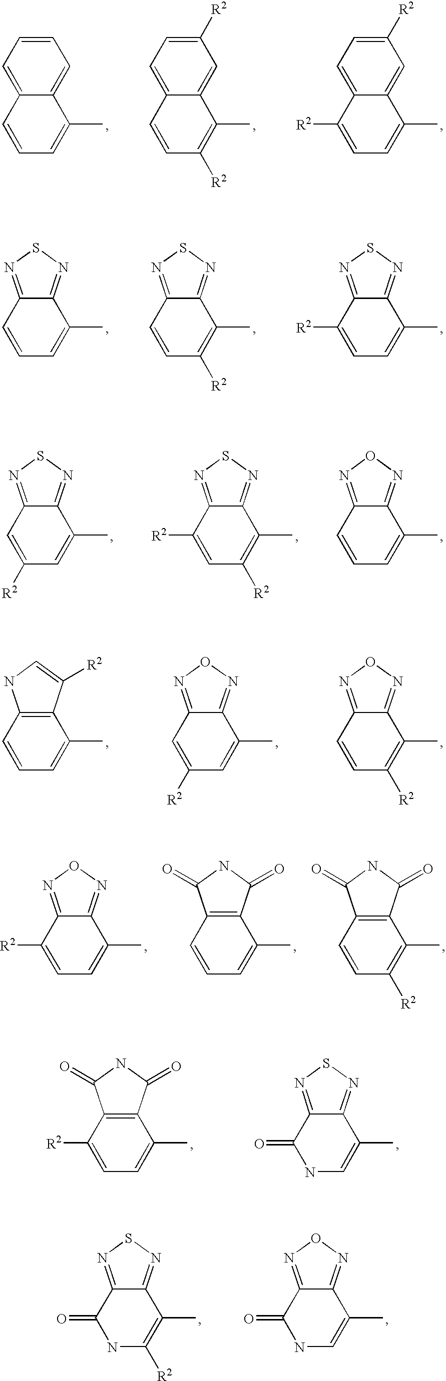 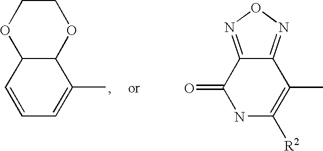
wherein R² has the meaning defined above.

Especially preferably, Z represents one of the following groups:

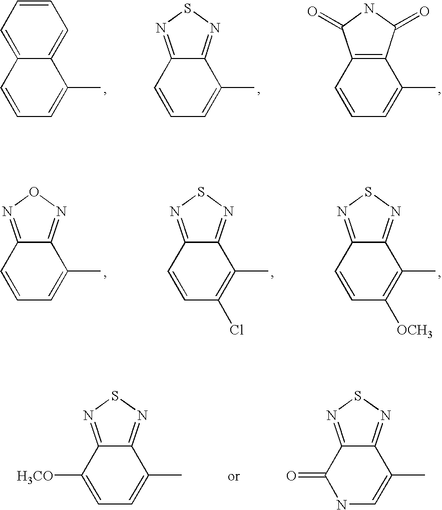

Halogen is preferably F, Cl, Br or I. F and Cl are espcially preferred.

n is preferably 1, 2 or 3. n is especially preferably 2.

The substituents R¹, R², X, Z, A and n can independently of one another assume one of the above mentioned meanings. The compounds of the general formula I are thus all the more strongly preferred, the more of their substituents have preferred meanings and the greater these meanings are preferred.

Compounds selected from the following group of the compounds Ia to Ik are particularly preferred: 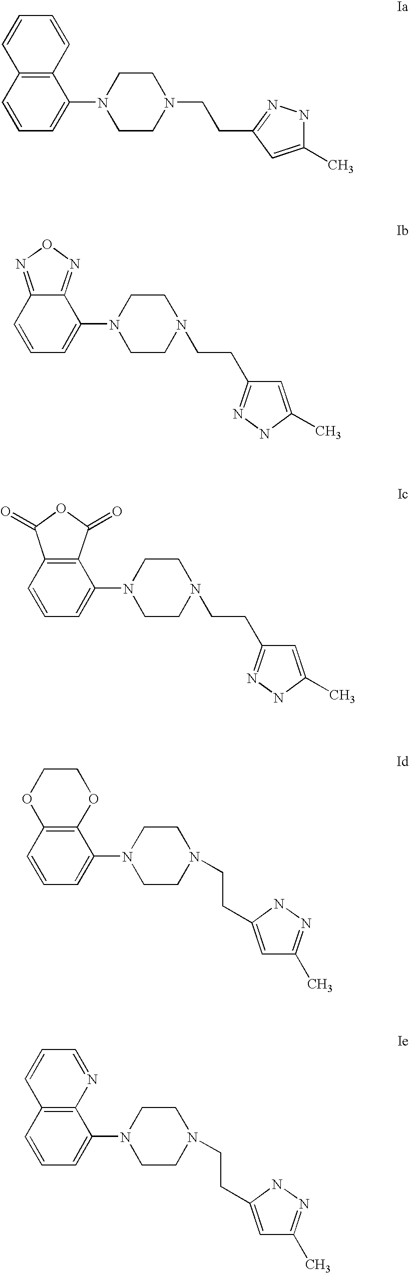 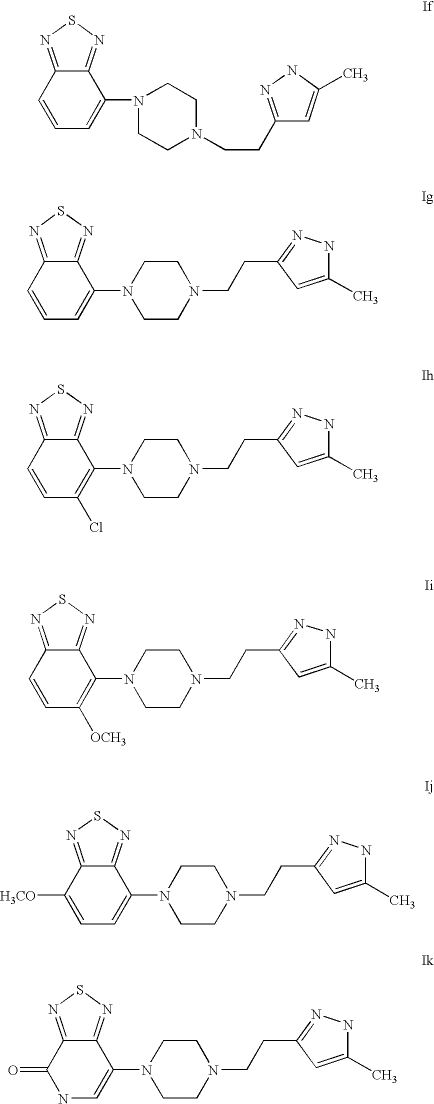
and their salts or solvates and solvates.

If the compounds of the general formula I are optically active, the formula I includes both any isolated optical antipodes and the corresponding optionally racemic mixtures in any conceivable composition.

A compound of the general formula I can be converted into the corresponding salt (that is acid addition salt) using an acid. Acids which afford the tolerable (that is biocompatible and adequately bioavailable) salts or solvates are suitable for this reaction. It is thus possible to use inorganic acids such as sulfuric acid or hydrohalic acids such as hydrochloric acid, bromic acid or phosphoric acids such as orthophosphoric acid, nitric acid, sulfamic acid, aliphatic, alicyclic, araliphatic, aromatic or heterocyclic monobasic or polybasic carboxylic acids, sulfonic acids or sulfuric acid derivatives such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methanesulfonic acid or ethanesulfonic acid, ethanedisulfonic acid, 2-hydroxyethanesulfonic acid, benzenesulfonic acid, paratoluenesulfonic acid, naphthalenemonosulfonic acid and naphthalenedisulfonic acid and sulfuric acid lauryl ester in order to obtain the corresponding acid addition salt.

If desired, the corresponding free bases of the general formula I can be liberated by the treatment of their salts or solvates with strong bases such as sodium hydroxide, potassium hydroxide or sodium or potassium carbonate, provided that no other acidic groups are present in the molecule. In the last-mentioned cases, in which the compounds of the general formula I carry free acidic groups, salt formation can also be brought about by treatment with strong bases. Suitable bases are alkali metal hydroxides, alkaline earth metal hydroxides, or organic bases in the form of primary, secondary or tertiary amines.

Solvates of the compounds of the general formula I are understood as meaning adducts of solvent molecules to the compounds of the formula I which are formed on account of their mutual attractive force. Solvates are, for example, mono- and dihydrates or addition compounds with alcohols such as methanol or ethanol.

It is known that pharmaceuticals can be converted synthetically into derivatives (for example into alkyl or acyl derivatives, into sugar or oligopeptide derivatives and others) which are converted back into the active compounds of the general formula I in the body metabolically by extracellular or intracellular enzymes. The invention also relates to such “prodrug derivatives” of the compounds of the general formula I.

A further subject of the invention is the use of a compound of the general formula I or of one of its tolerable salts or solvates for the production of a medicament which is suitable for the treatment of human or animal disorders, in particular of disorders of the central nervous system such as pathological stress states, depression and/or psychoses, for the reduction of side effects during the treatment of high blood pressure (e.g. with α-methyldopa), for the treatment of endocrinological and/or gynaecological disorders, e.g. for the treatment of acromegaly, hypogonadism, secondary amenorrhoea, the post-menstrual syndrome and undesired lactation in puberty and for the prophylaxis and therapy of cerebral disorders (e.g. of migraine), in particular in geriatrics, in a similar manner to specific ergot alkaloids and for the control and prophylaxis of cerebral infarct (cerebral apoplexy) such as cerebral stroke and cerebral ischaemia. Moreover, the pharmaceutical preparations and medicaments which contain a compound of the general formula I are suitable for improvement of the cognitive functional ability and for the treatment of Alzheimer's disease symptoms. In particular, such medicaments are suitable for the treatment of mental disorders of the schizophrenia type and for the control of psychotic anxiety states. The term treatment in the context of the invention includes prophylaxis and therapy of human or animal diseases.

The substances of the general formula I are normally administered analogously to known, commercially obtainable pharmaceutical preparations (e.g. of bromocriptine and dihydroergocornine), preferably in doses of between 0.2 and 500 mg, in particular of between 0.2 and 15 mg per dose unit. The daily dose unit is between 0.001 and 10 mg per kg of body weight. Low doses (of between 0.2 and 1 mg per dose unit, 0.001 to 0.005 mg per kg of body weight) are particularly suitable for pharmaceutical preparations for the treatment of migraine. A dose of between 10 and 50 mg per dose unit is preferred for other indications. However, the dose to be administered depends on a large number of factors, e.g. on the efficacy of the corresponding component, the age, the body weight and the general condition of the patient.

The invention also relates to the compounds of the formula I and their physiologically acceptable salts or solvates as pharmaceutical active compounds.

The invention furthermore relates to compounds of the formula I and their physiologically acceptable salts or solvates as D₂ receptor antagonists and 5-HT_{1A} agonists.

Moreover, the invention relates to compounds of the formula and/or their physiologically acceptable salts and/or solvates for the treatment or prophylaxis of diseases which can be combated or influenced by compounds having D₂ receptor antagonistic and/or 5-HT_{1A} agonistic properties.

The invention also relates to the compounds of the formula and their physiologically acceptable salts or solvates for use in the control of diseases.

A further subject of the invention is a process for the production of a pharmaceutical preparation, which comprises the conversion of a compound of the general formula I or of one of its tolerable salts or solvates to a suitable dose form together with a suitable vehicle. The compounds of the general formula I can be brought into a suitable dose form together with at least one vehicle or excipient, if appropriate in combination with a further active ingredient.

Suitable vehicles are organic or inorganic substances which are suitable for enteral (e.g. oral) or parenteral or topical administration and which do not react with the substances of the general formula I according to the invention. Examples of such vehicles are water, vegetable oils, benzyl alcohols, polyethylene glycols, gelatin, carbohydrates such as lactose and starch, magnesium stearate, talc and raw petroleum jelly. Tablets, coated tablets, capsules, syrups, juices, drops or suppositories are in particular employed for enteral administration. Solutions, preferably oily or aqueous solutions, such as suspensions, emulsions or alternatively implants are used for parenteral administration. Ointments, creams or powders are employed in the case of external application. The compounds of the general formula I can also be lyophilized and the resulting lyophilizates processed to give injectable preparations.

The invention further relates to medicaments which contain at least one compound of the general formula I or one of its tolerable salts or solvates and, if appropriate, further ingredients such as vehicles, excipients etc. These preparations can be employed as medicaments for the treatment of human or animal diseases.

The aforementioned medicaments can be sterilized and processed together with excipients such as lubricants, preservatives, stabilizers and/or wetting agents, emulsifiers, osmotically active substances, buffers, colorants or flavor enhancers to give other pharmaceutical preparations.

A further subject of the invention is a process for the preparation of compounds of the formula I, and their salts or solvates, characterized in that a compound of the formula II 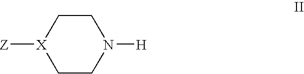
in which Z and X have the meanings indicated above,
is reacted with a compound of the formula III 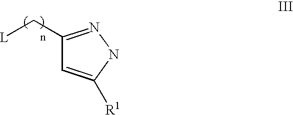
in which R¹ and n have the meanings indicated above and L is a leaving group, in particular Cl, tosylate or Br and, if appropriate, a basic or acidic compound of the formula I is converted into one of its salts or solvates by treating with an acid or base.

The compounds of the formula I and also the starting substances for their preparation are otherwise prepared by methods known per se, such as are described in the literature (e.g. in the standard works such as Houben-Weyl, Methoden der organischen Chemie [Methods of organic chemistry], Georg-Thieme-Verlag, Stuttgart), namely under reaction conditions which are known and suitable for the reactions mentioned. Use can also be made in this case of variants which are known per se, but not mentioned here in greater detail.

If desired, the starting substances can also be formed in situ such that they are not isolated from the reaction mixture, but immediately reacted further to give the compounds of the formula I.

The pyrazole derivatives of the formula I are preferably prepared according to the following scheme: 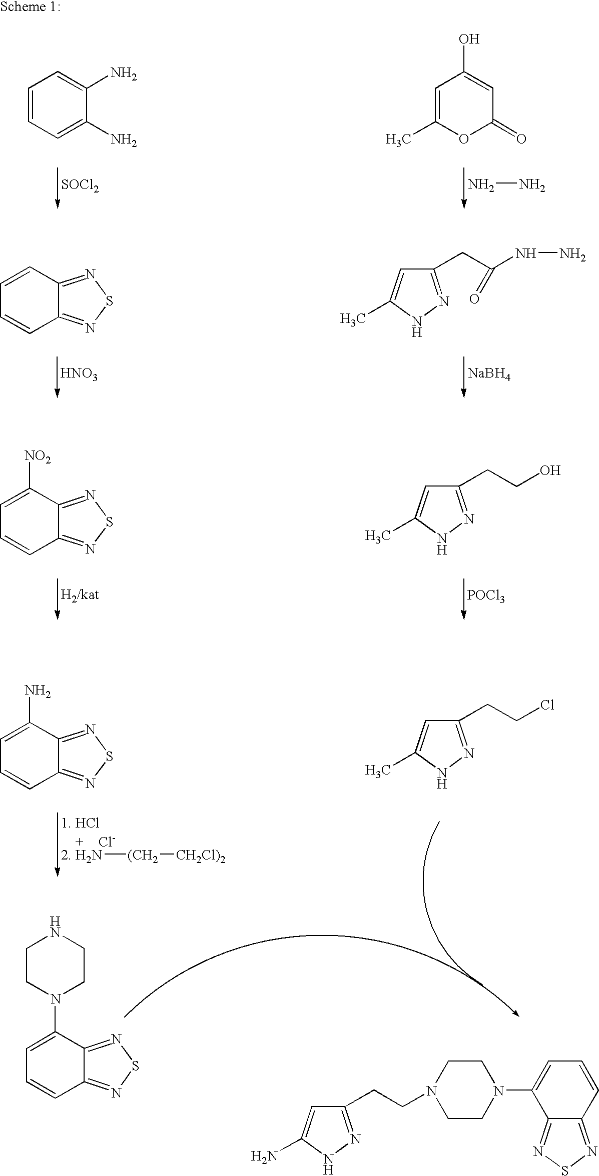
in which A, R¹ and R² have the meanings indicated above.

The invention is described by the following examples.

The molecular weight (M+H⁺) is determined with the aid of electron spray ionization mass spectroscopy. The mass-spectroscopic data derive from HPLC/IMSC runs (HPLC coupled with an electrospray ionization mass spectrometer). The numerical values are, as customary in this procedure, not the molecular weights of the unmodified compounds, but the molecular weights of the protonated compounds (below: [M+H⁺]). The method is described in the following references: M. Yamashita, J. B. Fenn, J. Phys. Chem. 88,1984, 4451-4459; C. K. Meng et al., Zeitschrift für Physik D 10, 1988, 361-368; J. B. Fenn et al., Science 246, 1989, 64-71.

### EXAMPLE 1

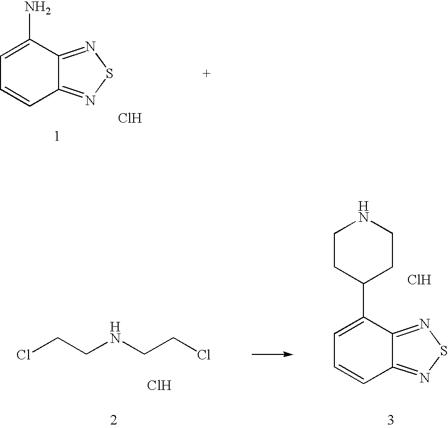

A solution of 142.8 g 2 in 200 ml of 1-Methyl-2-pyrrolidon is added dropwise to a solution of 60.0 g 1 in 200 ml of 1-Methyl-2-pyrrolidon. The mixture is stirred over night. Compound 3 is obtained by conventional work-up (m.p. 238-241° C.).

### EXAMPLE 2

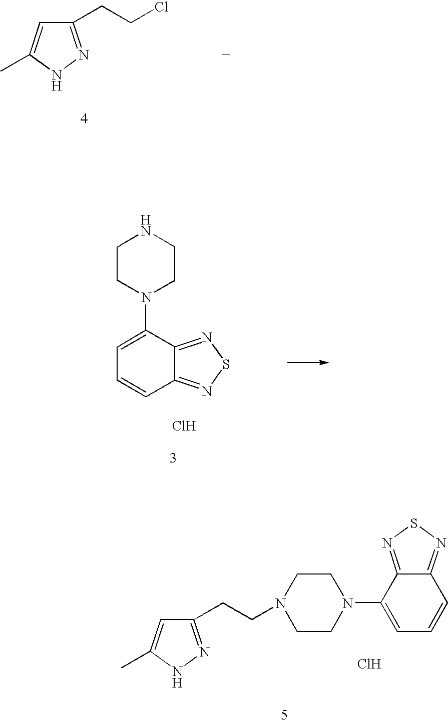

A mixture of 1.44 g 4 and 2.56 g 3 and 2.52 g sodium hydrogencarbonate is suspended in 20 ml of acetonitril and heated under reflux for 52 h. After cooling, the mixture is rendered alkaline and worked up conventionally. 5 is obtained after treatment of the resultant oil with hydrochloric acid. (m.p.198-201° C.).

### EXAMPLE 3

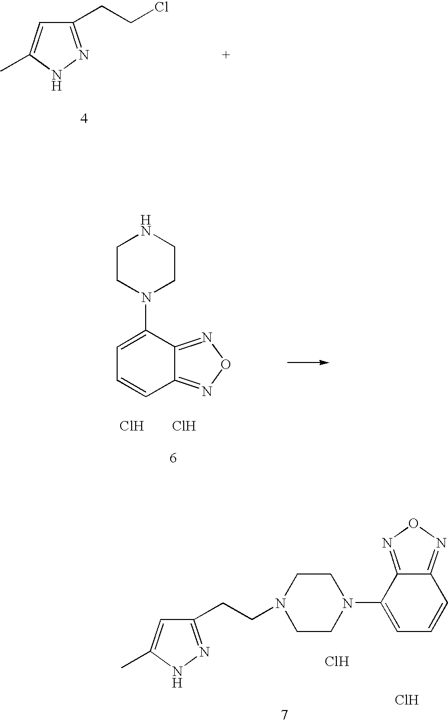

A mixture of 0.390 g 4, 0.750 g 6, 0.91 g sodium hydrogencarbonate and 10 ml acetonitril is heated under reflux and worked up as described in Example 2, whereby Z is obtained (m.p. 248-250° C.).

### EXAMPLE 4

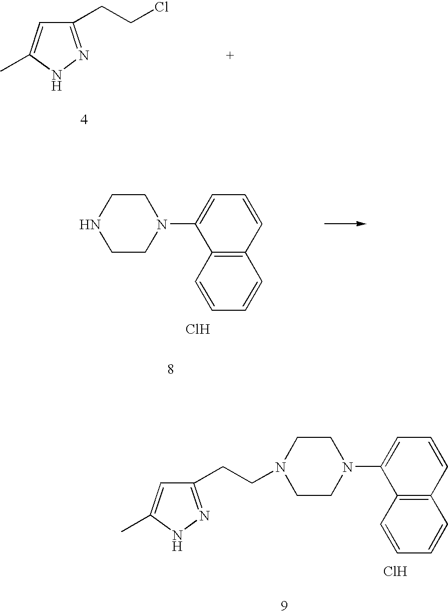

A mixture of 0.520 g 4, 0.900 g 8, 0.91 g sodium hydrogencarbonate and 20 ml acetonitril is heated under reflux and worked up as described in Example 2, whereby 9 is obtained (m.p. 257-259° C.).

### EXAMPLE 5

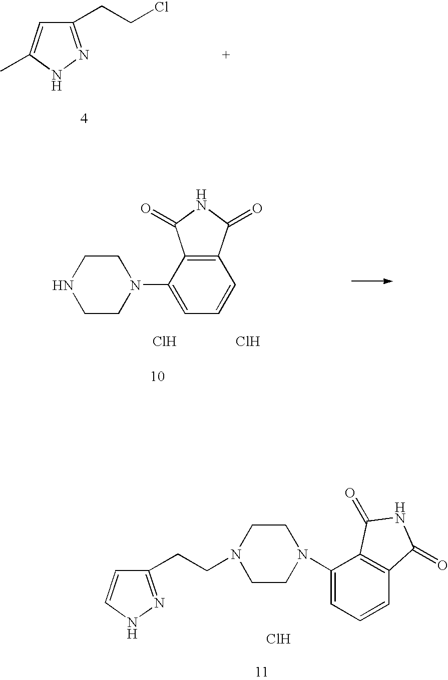

A mixture of 1.290 g 4, 2.800 g 10, 2.99 g sodium hydrogencarbonate and 250 ml acetonitril is heated under reflux and worked up as described in Example 2, whereby 11 is obtained (m.p. 221-223° C.).

The following compounds and their acid addition salts or solvates are prepared analogously using the appropriate precursors:

### EXAMPLES 6-34:

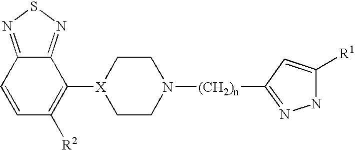

| | R¹ | R² | X | n |
|---|---|---|---|---|
| (6) | methyl | Br | N | 2 |
| (7) | methyl | methyl | N | 2 |
| (8) | methyl | ethyl | N | 2 |
| (9) | methyl | methoxy | N | 2 |
| (10) | methyl | ethoxy | N | 2 |
| (11) | methyl | CF₃ | N | 2 |
| (12) | methyl | OCF₃ | N | 2 |
| (13) | methyl | Cl | N | 2 |
| (14) | methyl | F | N | 2 |
| (15) | ethyl | Br | N | 2 |
| (16) | ethyl | methyl | N | 2 |
| (17) | ethyl | ethyl | N | 2 |
| (18) | ethyl | methoxy | N | 2 |
| (19) | ethyl | ethoxy | N | 2 |
| (20) | ethyl | CF₃ | N | 2 |
| (21) | ethyl | OCF₃ | N | 2 |
| (22) | ethyl | Cl | N | 2 |
| (23) | ethyl | F | N | 2 |
| (24) | methyl | H | CH | 2 |
| (25) | methyl | methyl | CH | 2 |
| (26) | methyl | ethyl | CH | 2 |
| (27) | methyl | methoxy | CH | 2 |
| (28) | methyl | ethoxy | CH | 2 |
| (29) | methyl | CF₃ | CH | 2 |
| (30) | methyl | OCF₃ | OH | 2 |
| (31) | methyl | Cl | CH | 2 |
| (32) | methyl | F | OH | 2 |
| (33) | methyl | H | N | 3 |
| (34) | methyl | H | N | 4 |

### EXAMPLES 35-63

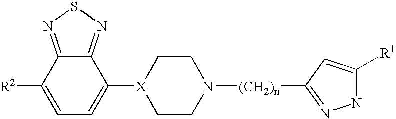

| | R¹ | R² | X | n |
|---|---|---|---|---|
| (35) | methyl | Br | N | 2 |
| (36) | methyl | methyl | N | 2 |
| (37) | methyl | ethyl | N | 2 |
| (38) | methyl | methoxy | N | 2 |
| (39) | methyl | ethoxy | N | 2 |
| (40) | methyl | CF₃ | N | 2 |
| (41) | methyl | OCF₃ | N | 2 |
| (42) | methyl | Cl | N | 2 |
| (43) | methyl | F | N | 2 |
| (44) | ethyl | Br | N | 2 |
| (45) | ethyl | methyl | N | 2 |
| (46) | ethyl | ethyl | N | 2 |
| (47) | ethyl | methoxy | N | 2 |
| (48) | ethyl | ethoxy | N | 2 |
| (49) | ethyl | CF₃ | N | 2 |
| (50) | ethyl | OCF₃ | N | 2 |
| (51) | ethyl | Cl | N | 2 |
| (52) | ethyl | F | N | 2 |
| (53) | methyl | Br | CH | 2 |
| (54) | methyl | methyl | CH | 2 |
| (55) | methyl | ethyl | CH | 2 |
| (56) | methyl | methoxy | CH | 2 |
| (57) | methyl | ethoxy | CH | 2 |
| (58) | methyl | CF₃ | CH | 2 |
| (59) | methyl | OCF₃ | CH | 2 |
| (60) | methyl | Cl | CH | 2 |
| (61) | methyl | F | CH | 2 |
| (62) | methyl | H | N | 3 |
| (63) | methyl | H | N | 4 |

### EXAMPLES 64-92

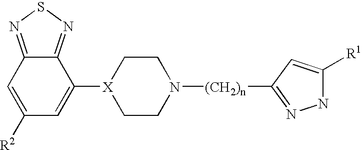

| | R¹ | R² | X | n |
|---|---|---|---|---|
| (64) | methyl | Br | N | 2 |
| (65) | methyl | methyl | N | 2 |
| (66) | methyl | ethyl | N | 2 |
| (67) | methyl | methoxy | N | 2 |
| (68) | methyl | ethoxy | N | 2 |
| (69) | methyl | CF₃ | N | 2 |
| (70) | methyl | OCF₃ | N | 2 |
| (71) | methyl | Cl | N | 2 |
| (72) | methyl | F | N | 2 |
| (73) | ethyl | Br | N | 2 |
| (74) | ethyl | methyl | N | 2 |
| (75) | ethyl | ethyl | N | 2 |
| (76) | ethyl | methoxy | N | 2 |
| (77) | ethyl | ethoxy | N | 2 |
| (78) | ethyl | CF₃ | N | 2 |
| (79) | ethyl | OCF₃ | N | 2 |
| (80) | ethyl | Cl | N | 2 |
| (81) | ethyl | F | N | 2 |
| (82) | methyl | H | CH | 2 |
| (83) | methyl | methyl | CH | 2 |
| (84) | methyl | ethyl | CH | 2 |
| (85) | methyl | methoxy | CH | 2 |
| (86) | methyl | ethoxy | CH | 2 |
| (87) | methyl | CF₃ | CH | 2 |
| (88) | methyl | OCF₃ | CH | 2 |
| (89) | methyl | Cl | CH | 2 |
| (90) | methyl | F | CH | 2 |
| (91) | methyl | H | N | 3 |
| (92) | methyl | H | N | 4 |

### EXAMPLES 93-121

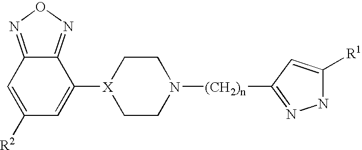

| | R¹ | R² | X | n |
|---|---|---|---|---|
| (93) | methyl | Br | N | 2 |
| (94) | methyl | methyl | N | 2 |
| (95) | methyl | ethyl | N | 2 |
| (96) | methyl | methoxy | N | 2 |
| (97) | methyl | ethoxy | N | 2 |
| (98) | methyl | CF₃ | N | 2 |
| (99) | methyl | OCF₃ | N | 2 |
| (100) | methyl | Cl | N | 2 |
| (101) | methyl | F | N | 2 |
| (102) | ethyl | Br | N | 2 |
| (103) | ethyl | methyl | N | 2 |
| (104) | ethyl | ethyl | N | 2 |
| (105) | ethyl | methoxy | N | 2 |
| (106) | ethyl | ethoxy | N | 2 |
| (107) | ethyl | CF₃ | N | 2 |
| (108) | ethyl | OCF₃ | N | 2 |
| (109) | ethyl | Cl | N | 2 |
| (110) | ethyl | F | N | 2 |
| (111) | methyl | H | CH | 2 |
| (112) | methyl | methyl | CH | 2 |
| (113) | methyl | ethyl | CH | 2 |
| (114) | methyl | methoxy | CH | 2 |
| (115) | methyl | ethoxy | CH | 2 |
| (116) | methyl | CF₃ | CH | 2 |
| (117) | methyl | OCF₃ | CH | 2 |
| (118) | methyl | Cl | CH | 2 |
| (119) | methyl | F | CH | 2 |
| (120) | methyl | H | N | 3 |
| (121) | methyl | H | N | 4 |

### EXAMPLES 122-150

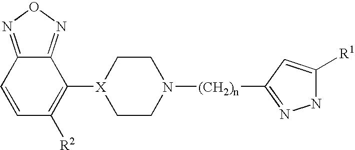

| | R¹ | R² | X | n |
|---|---|---|---|---|
| (122) | methyl | r | N | 2 |
| (123) | methyl | methyl | N | 2 |
| (124) | methyl | ethyl | N | 2 |
| (125) | methyl | methoxy | N | 2 |
| (126) | methyl | ethoxy | N | 2 |
| (127) | methyl | CF₃ | N | 2 |
| (128) | methyl | OCF₃ | N | 2 |
| (129) | methyl | Cl | N | 2 |
| (130) | methyl | F | N | 2 |
| (131) | ethyl | Br | N | 2 |
| (132) | ethyl | methyl | N | 2 |
| (133) | ethyl | ethyl | N | 2 |
| (134) | ethyl | methoxy | N | 2 |
| (135) | ethyl | ethoxy | N | 2 |
| (136) | ethyl | CF₃ | N | 2 |
| (137) | ethyl | OCF₃ | N | 2 |
| (138) | ethyl | Cl | N | 2 |
| (139) | ethyl | F | N | 2 |
| (140) | methyl | Br | CH | 2 |
| (141) | methyl | methyl | CH | 2 |
| (142) | methyl | ethyl | CH | 2 |
| (143) | methyl | methoxy | CH | 2 |
| (144) | methyl | ethoxy | CH | 2 |
| (145) | methyl | CF₃ | CH | 2 |
| (146) | methyl | OCF₃ | CH | 2 |
| (147) | methyl | Cl | CH | 2 |
| (148) | methyl | F | CH | 2 |
| (149) | methyl | H | N | 3 |
| (150) | methyl | H | N | 4 |

### EXAMPLES 151-179

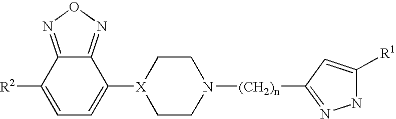

| | R¹ | R² | X | n |
|---|---|---|---|---|
| (151) | methyl | Br | N | 2 |
| (152) | methyl | methyl | N | 2 |
| (153) | methyl | ethyl | N | 2 |
| (154) | methyl | methoxy | N | 2 |
| (155) | methyl | ethoxy | N | 2 |
| (156) | methyl | CF₃ | N | 2 |
| (157) | methyl | OCF₃ | N | 2 |
| (158) | methyl | Cl | N | 2 |
| (159) | methyl | F | N | 2 |
| (160) | methyl | Br | N | 2 |
| (161) | ethyl | methyl | N | 2 |
| (162) | ethyl | ethyl | N | 2 |
| (163) | ethyl | methoxy | N | 2 |
| (164) | ethyl | ethoxy | N | 2 |
| (165) | ethyl | CF₃ | N | 2 |
| (166) | ethyl | OCF₃ | N | 2 |
| (167) | ethyl | Cl | N | 2 |
| (168) | ethyl | F | N | 2 |
| (169) | methyl | Br | CH | 2 |
| (170) | methyl | methyl | CH | 2 |
| (171) | methyl | ethyl | CH | 2 |
| (172) | methyl | methoxy | CH | 2 |
| (173) | methyl | ethoxy | CH | 2 |
| (174) | methyl | CF₃ | CH | 2 |
| (175) | methyl | OCF₃ | CH | 2 |
| (176) | methyl | Cl | CH | 2 |
| (177) | methyl | F | CH | 2 |
| (178) | methyl | H | N | 3 |
| (179) | methyl | H | N | 4 |

### EXAMPLES 180-208

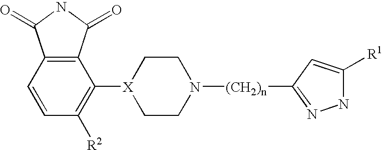

| | R¹ | R² | X | n |
|---|---|---|---|---|
| (180) | methyl | Br | N | 2 |
| (181) | methyl | methyl | N | 2 |
| (182) | methyl | ethyl | N | 2 |
| (183) | methyl | methoxy | N | 2 |
| (184) | methyl | ethoxy | N | 2 |
| (185) | methyl | CF₃ | N | 2 |
| (186) | methyl | OCF₃ | N | 2 |
| (187) | methyl | Cl | N | 2 |
| (188) | methyl | F | N | 2 |
| (189) | ethyl | Br | N | 2 |
| (190) | ethyl | methyl | N | 2 |
| (191) | ethyl | ethyl | N | 2 |
| (192) | ethyl | methoxy | N | 2 |
| (193) | ethyl | ethoxy | N | 2 |
| (194) | ethyl | CF₃ | N | 2 |
| (195) | ethyl | OCF₃ | N | 2 |
| (196) | ethyl | Cl | N | 2 |
| (197) | ethyl | F | N | 2 |
| (198) | methyl | H | CH | 2 |
| (199) | methyl | methyl | CH | 2 |
| (200) | methyl | ethyl | CH | 2 |
| (201) | methyl | methoxy | CH | 2 |
| (202) | methyl | ethoxy | CH | 2 |
| (203) | methyl | CF₃ | CH | 2 |
| (204) | methyl | OCF₃ | CH | 2 |
| (205) | methyl | Cl | CH | 2 |
| (206) | methyl | F | CH | 2 |
| (207) | methyl | H | N | 3 |
| (208) | methyl | H | N | 4 |

### EXAMPLES 209-237

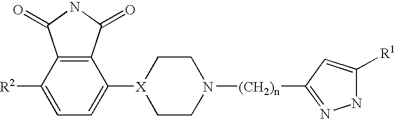

| | R¹ | R² | X | n |
|---|---|---|---|---|
| (209) | methyl | Br | N | 2 |
| (210) | methyl | methyl | N | 2 |
| (211) | methyl | ethyl | N | 2 |
| (212) | methyl | methoxy | N | 2 |
| (213) | methyl | ethoxy | N | 2 |
| (214) | methyl | CF₃ | N | 2 |
| (215) | methyl | OCF₃ | N | 2 |
| (216) | methyl | Cl | N | 2 |
| (217) | methyl | F | N | 2 |
| (218) | ethyl | Br | N | 2 |
| (219) | ethyl | methyl | N | 2 |
| (220) | ethyl | ethyl | N | 2 |
| (221) | ethyl | methoxy | N | 2 |
| (222) | ethyl | ethoxy | N | 2 |
| (223) | ethyl | CF₃ | N | 2 |
| (224) | ethyl | OCF₃ | N | 2 |
| (225) | ethyl | Cl | N | 2 |
| (226) | ethyl | F | N | 2 |
| (227) | methyl | Br | CH | 2 |
| (228) | methyl | methyl | CH | 2 |
| (229) | methyl | ethyl | CH | 2 |
| (230) | methyl | methoxy | CH | 2 |
| (231) | methyl | ethoxy | CH | 2 |
| (232) | methyl | CF₃ | CH | 2 |
| (233) | methyl | OCF₃ | CH | 2 |
| (234) | methyl | Cl | CH | 2 |
| (235) | methyl | F | CH | 2 |
| (236) | methyl | H | N | 3 |
| (237) | methyl | H | N | 4 |

### EXAMPLES 238-266

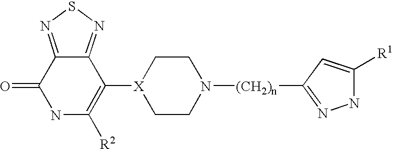

| | R¹ | R² | X | n |
|---|---|---|---|---|
| (238) | methyl | H | N | 2 |
| (239) | methyl | methyl | N | 2 |
| (240) | methyl | ethyl | N | 2 |
| (241) | methyl | methoxy | N | 2 |
| (242) | methyl | ethoxy | N | 2 |
| (243) | methyl | CF₃ | N | 2 |
| (244) | methyl | OCF₃ | N | 2 |
| (245) | methyl | Cl | N | 2 |
| (246) | methyl | F | N | 2 |
| (247) | ethyl | H | N | 2 |
| (248) | ethyl | methyl | N | 2 |
| (249) | ethyl | ethyl | N | 2 |
| (250) | ethyl | methoxy | N | 2 |
| (251) | ethyl | ethoxy | N | 2 |
| (252) | ethyl | CF₃ | N | 2 |
| (253) | ethyl | OCF₃ | N | 2 |
| (254) | ethyl | Cl | N | 2 |
| (255) | ethyl | F | N | 2 |
| (256) | methyl | H | CH | 2 |
| (257) | methyl | methyl | CH | 2 |
| (258) | methyl | ethyl | CH | 2 |
| (259) | methyl | methoxy | CH | 2 |
| (260) | methyl | ethoxy | CH | 2 |
| (261) | methyl | CF₃ | CH | 2 |
| (262) | methyl | OCF₃ | CH | 2 |
| (263) | methyl | Cl | CH | 2 |
| (264) | methyl | F | CH | 2 |
| (265) | methyl | H | N | 3 |
| (266) | methyl | H | N | 4 |

### EXAMPLES 267-295

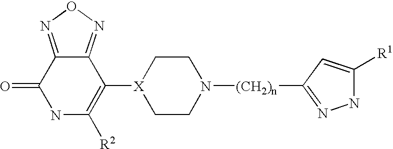

| | R¹ | R² | X | n |
|---|---|---|---|---|
| (267) | methyl | H | N | 3 |
| (268) | methyl | methyl | N | 2 |
| (269) | methyl | ethyl | N | 2 |
| (270) | methyl | methoxy | N | 2 |
| (271) | methyl | ethoxy | N | 2 |
| (272) | methyl | CF₃ | N | 2 |
| (273) | methyl | OCF₃ | N | 2 |
| (274) | methyl | Cl | N | 2 |
| (275) | methyl | F | N | 2 |
| (276) | ethyl | H | N | 4 |
| (277) | ethyl | methyl | N | 2 |
| (278) | ethyl | ethyl | N | 2 |
| (279) | ethyl | methoxy | N | 2 |
| (280) | ethyl | ethoxy | N | 2 |
| (281) | ethyl | CF₃ | N | 2 |
| (282) | ethyl | OCF₃ | N | 2 |
| (283) | ethyl | Cl | N | 2 |
| (284) | ethyl | F | N | 2 |
| (285) | methyl | H | CH | 2 |
| (286) | methyl | methyl | CH | 2 |
| (287) | methyl | ethyl | CH | 2 |
| (288) | methyl | methoxy | CH | 2 |
| (289) | methyl | ethoxy | CH | 2 |
| (290) | methyl | CF₃ | CH | 2 |
| (291) | methyl | OCF₃ | CH | 2 |
| (292) | methyl | Cl | CH | 2 |
| (293) | methyl | F | CH | 2 |
| (294) | methyl | H | N | 3 |
| (295) | methyl | H | N | 4 |

### EXAMPLE A

Ampoules for Injection

A solution of 100 g of a compound of the general formula I and 5 g of disodium hydrogenphosphate is adjusted to pH 6.5 using 2 N hydrochloric acid in 3 l of double-distilled water, sterile filtered and filled into injection ampoules, and lyophilized. Sterile conditions were adhered to here. Each injection ampoule contains 5 mg of the active component of the general formula I.

### EXAMPLE B

A mixture of 20 g of a compound of the general formula I is mixed with 100 g of soya lecithin and 1400 g of cocoa butter with warming and poured into hollows. Each suppository contains 20 mg of the active component.

### EXAMPLE C

A solution comprising 1 g of a compound of the general formula I, 9.38 g of NaH₂PO₄×2 H₂O, 28.48 g of Na₂HPO₄×12 H₂O and 0.1 g of benzalkonium chloride is prepared using 940 ml of double-distilled water. The solution is adjusted to pH 6.8 and made up to one litre with double-distilled water and sterilized by irradiation. This solution can be used in the form of eye drops.

### EXAMPLE D

Ointment

500 mg of a compound of the general formula I are blended with 99.5 g of raw petroleum jelly under aseptic conditions.

### EXAMPLE E

Tablets

100 g of a compound of the general formula I, 1 kg of lactose, 600 g of microcrystalline cellulose, 600 g of cornstarch, 100 g of polyvinyl-pyrrolidone, 80 g of talc and 10 g of magnesium stearate are mixed and pressed in a customary manner to give tablets such that one tablet contains 100 mg of the active component.

### EXAMPLE F

Coated Tablets

Tablets are prepared as in Example 7 and then coated in a known manner with sucrose, maize starch, talc, tragacanth gum and colorants.

### EXAMPLE G

Capsules

Hard gelatin capsules are filled with a compound of the general formula I in a known manner such that each capsule contains 5 mg of the active component.

### EXAMPLE H

Inhalation Spray

14 g of a compound of the general formula I are dissolved in 10 l of isotonic saline solution. The solution is filled into commercially obtainable spray containers which have a pump mechanism. The solution can be sprayed into the mouth or into the nose. One puff of spray (approximately 0.1 ml) corresponds to a dose of 0.14 mg of a compound of the general formula I.

1. Pyrazole derivatives of the formula I 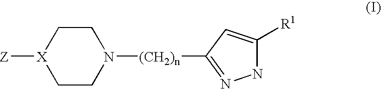
where
x is N or CH,
Z is 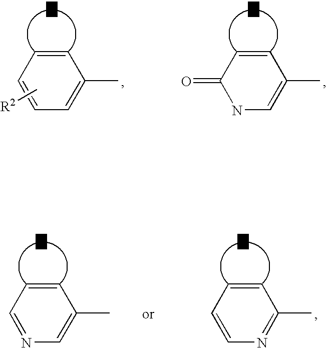
A is an aromatic or aliphatic ring wherein one or more CH-groups may be replaced by N or CR², or wherein one or more CH₂-groups may be replaced by NH, CO, SO, SO₂, S or O,
R¹ is H, or alkyl having 1 to 10 C-atoms,
R² is H, Halogen or alkyl or alkoxy having 1 to 10 C-Atoms, wherein one or more H-atoms may be replaced by F,
and
n is 1, 2, 3 or 4
and their salts and solvates.

2. Compounds of the formula I according to claim 1, characterized in that the group Z has one of the following meanings:

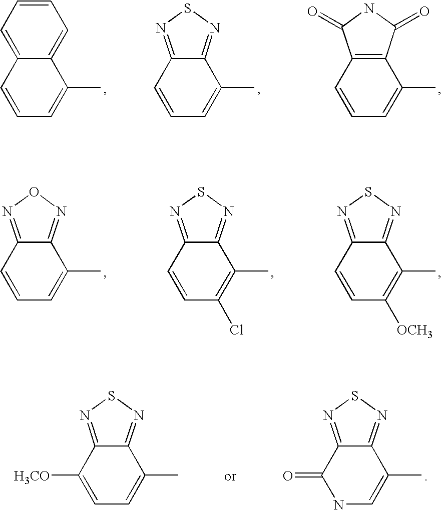

3. Compounds of the formula I according to claim 1, characterized in that X is N.

4. Compounds of the formula I according to claim 1, characterized in that R¹ is methyl.

5. Compounds of the formula I according to claim 1, characterized in that n is 2.

6. Compounds selected from the following group of compounds Ia to Ik: 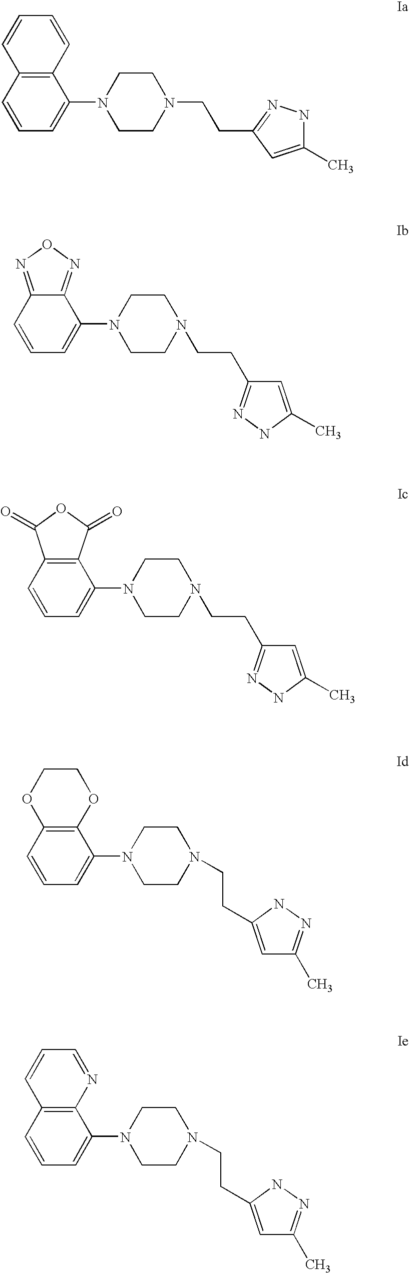 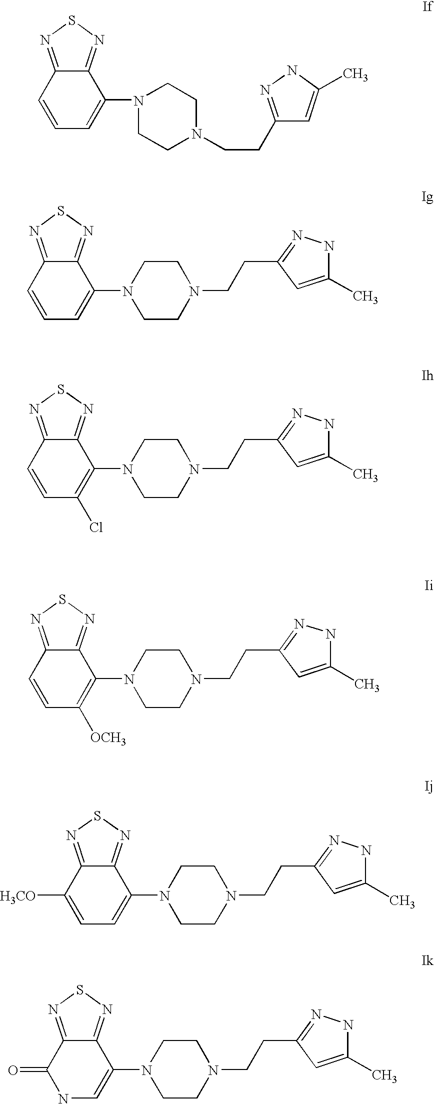
and their salts or solvates and solvates.

7. Compounds of the formula I according to claim 1 and their physiologically acceptable salts or solvates as pharmaceutical active compounds.

8. Compounds of the formula I according to claim 1 and their physiologically acceptable salts or solvates as D₂ receptor antagonists and/or 5-HT_{1A} agonists.

9. Compounds of the formula I according to claim 1 and/or their physiologically acceptable salts and/or solvates for the treatment or prophylaxis of diseases which can be combated or influenced by compounds having D₂ receptor antagonistic and/or 5-HT_{1A} agonistic properties.

10. Compounds of the formula I according to claim 1 and their physiologically acceptable salts or solvates for use in the control of diseases.

11. Pharmaceutical preparation characterized in that it contains at least one compound of the formula I according to claim 1 and/or one of its physiologically acceptable salts and/or solvates.

12. Use of compounds of the formula I according to claim 1 and/or their physiologically acceptable salts and/or solvates for the production of a medicament.

13. Use of compounds according to claim 1 and/or their physiologically acceptable salts and/or solvates for the production of a medicament for the prophylaxis and treatment of extrapyramidal movement disorders and/or illnesses of the central nervous system.

14. Use of compounds according to claim 1 and/or their physiologically acceptable salts and/or solvates for the production of a medicament for the prophylaxis and treatment of depressions, Alzheimer disease, cerebral infarcts, overexcitation, hyperactivity, attention disorders, developmental disorders, compulsive disorders, sexual function disorders, sleep and eating disorders, mental disorders of the schizophrenia type and for the control of psychotic anxiety states, idiophathic Parkinson's disease, adverse effects of anti-Pakinsonian drugs in idiophathic Parkinson's disease, Parkinson syndromes, adverse effects of anti-Pakinsonian drugs in Parkinson syndromes, dyskinetic, choreatic and dystonic syndromes, extrapyramidal symptoms induced by neuroleptics, tremor, Gilles de la Tourette syndrome, ballism, myoclonus, restless legs syndrome and Wison's disease.

15. Pharmaceutical composition comprising, as active principles,
(i) at least one compound of the formula I and/or a physiologically acceptable salt and/or solvate thereof, and
(ii) at least one conventional anti-Parkinsonian drug, in combination with one or more pharmaceutically acceptable excipients.

16. Composition according to claim 15 for enhancing the anti-Parkinsonian effect of the anti-Parkinsonian drug.

17. Composition according to claim 15, in which
(i) the active principle is in the form of its hydrochloride and
(ii) the conventional anti-Parkinsonian drug is l-dopa.

18. Composition according to claim 15, in which
(i) the active principle is in the form of its hydrochloride and
(ii) the conventional anti-Parkinsonian drug is l-dopa combined with benserazide and/or carbidopa.

19. Use of the compounds of formula I and/or a physiologically acceptable salt and/or solvate thereof in combination with at least one anti-Parkinsonian drug, for the preparation of a medicinal combination which enhances the anti-Parkinsonian effect of conventional anti-Parkinsonian drugs.

20. Process for the preparation of compounds of the formula I and their salts and solvates, characterized in that a compound of the formula II 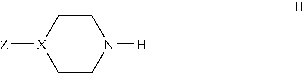
in which Z and X have the meanings indicated in claim 1,
is reacted with a compound of the formula III 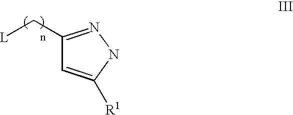
in which R¹ and n have the meanings indicated in claim 1 and L is a leaving group and, if appropriate, a basic or acidic compound of the formula I is converted into one of its salts or solvates by treating with an acid or base.

21. Compounds of the formula II 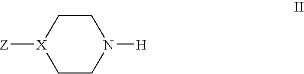
in which Z and X have the meanings indicated in claim 1.
